(19) United States
(12) Patent Application Publication
(10) Pub. No.: US 2003/0125688 A1
(43) Pub. Date: Jul. 3, 2003

(54) Adhesive system for mechanically post-treated absorbent structures

(76) Inventors: James M. Keane, Conway, AR (US); Andrew M. Lake, Manitowoc, WI (US); Steven J. Nielsen, Conway, AR (US); Shelley R. Rasmussen, Oshkosh, WI (US)

Correspondence Address: KIMBERLY-CLARK WORLDWIDE, INC., 401 NORTH LAKE STREET, NEENAH, WI, 54956

(21) Appl. No.: 10/306,546

(22) Filed: Nov. 26, 2002

Related U.S. Application Data
(60) Provisional application No. 60/336,097, filed on Nov. 30, 2001.

(51) Int. Cl. A61F 13/15

(52) U.S. Cl. 604/383; 604/385.01

(57) ABSTRACT

An absorbent structure that includes: (1) a liquid permeable cover made up of a matrix of fibrous materials where the matrix has upper and lower surfaces and a plurality individual exposed fiber surfaces at or adjacent with each surface of the matrix; (2) at least one layer of at least one liquid management material having a upper and lower surface; and (3) an adhesive system that joins at least a portion of the lower surface of the liquid permeable cover and the upper surface of the liquid management material.. The continuous layer of adhesive overlaying the permeable cover material desirably has a basis weight of 7.5 gsm or less. For example, layer of adhesive may have a basis weight of 4 gsm or 1.0 gsm or less .The absorbent structure may be mechanically post-treated. For example, the absorbent structure may be introduced into a nip and/or subjected to mechanical post treatments such as, for example, embossing, perforating, brushing, creping, aperturing and the like. A method of making an absorbent structure that may include the following steps: (1) providing at least a first fibrous material with an upper and lower surface and a second fibrous material with an upper and lower surface; (2) joining the lower surface of the first fibrous material and the upper surface of the second fibrous material with an adhesive to form an absorbent structure; and (3) mechanically post-treating the absorbent structure. A method of maintaining an aperture in a fibrous laminate which may include the steps of: (1) providing at least a first fibrous material with an upper and lower surface and a second fibrous material with an upper and lower surface; (2) joining the lower surface of the first fibrous material and the upper surface of the second fibrous material with an adhesive to form a fibrous laminate; and (3) aperturing the fibrous laminate such that at least one aperture passes through at least one of the fibrous materials and into the other fibrous material such that the adhesive that joins the tow fibrous materials is adapted to maintain the tow fibrous materials in position to define individual apertures.

This application claims priority from U.S. Provisional Application No. 60/336,097 filed Nov. 30, 2001.

### BACKGROUND OF THE INVENTION

The present invention relates to an adhesive system for absorbent structures. More particularly, the present invention relates to an adhesive system for an absorbent structure that is subjected to mechanical post-treatment.

Generally speaking, conventional absorbent personal care products contain an adhesive of some type to hold components or structures together during production and use. Many of these conventional products include one or more absorbent composite laminate materials. These absorbent composite laminate materials typically use adhesive, thermal and/or mechanical bonding to join layers of the absorbent composite laminate together and/or join the absorbent composite laminate to other components.

For example, at least one commercial product utilizes a pin stripe pattern of adhesive across the full width of the product to join a liquid permeable cover and absorbent materials immediately below the cover. Such an application of adhesive can generate many problems when mechanically post-treating (i.e., manipulating) the absorbent structure. These problems may be particularly evident when the assembly passes between a nip created by two or more rolls. While use of adhesives to join elements may be described in the literature, it appears that little, if any, references address the interaction between adhesives and subsequent mechanical post-treatment or manipulating steps.

For example, U.S. Pat. Nos. 5,411,497, 5,425,725, 5,433,715, and U.S. Pat. No. 5,593,399, assigned to Kimberly-Clark Corporation, discuss the use of a water sensitive adhesive for the formation of pockets between two carrier layers containing superabsorbent particles. The adhesive pattern can be a uniform continuous layer, patterned layer, swirls, spots, etc. According to at least some of these references, adhesive may be applied to one or both carrier sheets with an add-on amount greater than 7.5 grams per square meter and less than 150 grams per square meter.

U.S. Pat. No. 4,573,986, assigned to The Procter & Gamble Company, teaches the use of a pattern or network of open filaments or fibers between a liquid permeable cover and an absorbent element at an add-on level ranging from 0.8 to 4.7 grams per square meter. According to this patent, an open pattern or network of adhesive is used to join an absorbent core of relatively unbonded fibers to a permeable cover.

U.S. Pat. Nos. 4,069,822 and 4,147,580, assigned to The Procter & Gamble Company, teach a porous web that is overall or pattern coated with an extremely low level of hot-melt adhesive by causing the hot-melt adhesive to be wiped from an adhesive source by the individual projecting surface fibers and fiber junctions of the web to form globules on the individual projecting surfaces.

U.S. Pat. No. 5,560,974, assigned to Kappler Safety Group, discusses the use of a non-continuous application of adhesive to avoid interfering with the functionality of microporous films or film and nonwoven laminates.

U.S. Pat. No. 5,843,057, assigned to Kimberly-Clark Worldwide, Inc., teaches a pattern of adhesive for use in laminating a film and nonwoven material. The add-on detailed in between 0.1 and 20 grams per square meter.

U.S. Pat. No. 6,063,981, assigned to Kimberly-Clark Worldwide, Inc., discusses an adhesive used to prepare absorbent products without visible adhesive staining. This appears to relate to a breathable microporous film materials. The amount of adhesive may be, for example, between 0.5 and 10 grams per square meter of applied surface area.

U.S. Pat. No. 6,231,555, assigned to The Proctor and Gamble Company, teaches an adhesive of unspecified add-on level or rate, which is used to hold together layers of an absorbent article. This is done with a continuous pattern layer of meltblown fibers, separate lines, spirals, spots or the like.

Finally, U.S. H0001989, assigned to Kimberly-Clark Worldwide, Inc., discusses using adhesive on a microporous film to create regions of zoned breathability. The adhesive levels mentioned range from 1 to 7 grams per square meter. This document appears to describe the need to use an appropriate level of adhesive to obtain the desired breathability level in a product.

While these references disclose the use of adhesive with various materials and may describe how to manipulate the adhesive application to avoid interfering with certain physical properties of a structure, they fail to address the problems encountered when adhesives are used in absorbent structures that are mechanically post-treated (i.e., manipulated) in processes such as aperturing processes. This need is particularly apparent when the adhesively treated absorbent structure passes between a nip created by two or more aperturing rolls.

For example, some exemplary personal care products use a full width pin stripe adhesive pattern between a liquid permeable cover and the absorbent materials. Such a full width pattern allows for exposed adhesive to enter a nip situation or post treatment equipment such as an aperture module.

During the manufacture of some product forms, discrete pieces of absorbent material are placed on a continuous strip of liquid permeable cover material. Conventional applications of adhesives leave exposed adhesive on each side of the discretely placed absorbent component. The exposed adhesive may create increased web wrap-ups or cause adhesive build-up on post treatment equipment. For example, adhesive may cause hole plugging on aperture modules.

During some manufacturing operations in which a continuous strip of liquid permeable cover material and discrete absorbent components placed on the continuous strip are post-treated (i.e., treated after the discrete component is attached to the continuous strip), poor placement of adhesive can cause web wrap-ups, separation of the discrete attached components and the like. If the attached components are passed through an aperturing module, additional complications may be encountered. For example, if the aperturing module utilizes an aperture roll with protruding pins, the pin tip acceleration can distort hole quality and cause the web to wrap the pin roll in a clockwise fashion. Configuring the web assembly to have more contact with the female roll of the aperture module can cause additional contact with adhesive that can cause undesirable adhesive build-up and/or web-sticking to the roll.

While it might be possible to apply special coatings to surfaces that may be exposed to adhesive, this generally does not eliminate the problems. It might also be possible to mechanically treat the components prior to applying adhesive and joining them together. However, this sequence of steps creates problems with registration and, in the case of aperturing, destroys the clarity and visual cue provided by aperturing the components together. The resulting aperture holes were not aligned for the two materials and did not provide the same desirable appearance.

A related issue is the maintenance of apertures within the materials. This issue is especially prevalent with highly resilient materials and with co-apertured materials where the materials have differing resiliencies. In such situations, the resilient material has a tendency to recover over time after aperturing. As the aperture is lost, there is a corresponding loss of the performance and aesthetics that was originally given to the materials by aperturing.

### SUMMARY OF THE INVENTION

The present invention relates to adhesive application that prevents and/or eliminates the significant problems generated by adhesives in absorbent structures that are mechanically treated after adhesive is applied. This invention has particular importance in the production of personal care products which requires such absorbent structure manufacture.

Generally speaking, the invention relates to an absorbent structure that includes: (1) a liquid permeable cover made up of a matrix of fibrous materials where the matrix has upper and lower surfaces and a plurality individual exposed fiber surfaces at or adjacent with each surface of the matrix; (2) at least one layer of at least one liquid management material having a upper and lower surface; and (3) an adhesive system that joins at least a portion of the lower surface of the liquid permeable cover and the upper surface of the liquid management material.

According to the invention, the adhesive system is a thin, substantially continuous coating of adhesive on at least a portion of the plurality of individual exposed fiber surfaces. The overall amount of adhesive is low enough to avoid interfering with liquid transport through the liquid permeable cover and into the liquid management material. In other words, the amount of adhesive is low enough to essentially prevent formation of a hydrophobic barrier layer or liquid impermeable layer between the permeable cover and the liquid management material.

While the adhesive system is desirably a hot-melt adhesive, other adhesives such as, for example, aqueous adhesives and label-type adhesives may be used. Generally speaking, the adhesive should avoid form globules or globs on the surface of the permeable cover and should instead form a thin, generally continuous coating or film. The adhesive is desirably applied in such a generally thin, uniform film by utilizing slot coat techniques. Other application techniques that provide a thin, uniform continuous coating on the permeable cover material surface may be used.

The continuous layer of adhesive overlaying the permeable cover material desirably has a basis weight of 7.5 gsm or less. For example, layer of adhesive may have a basis weight of 4 gsm or less. As another example, the layer of adhesive may have a basis weight of 2.5 gsm or less. As yet another example, the layer of adhesive may have a basis weight of 1.5 gsm or less. As a further example, the layer of adhesive may have a basis weight of 1.0 gsm or less.

In one embodiment of the present invention, the continuous layer of adhesive effectively coats the plurality of individual exposed fiber surfaces such that in the area of adhesive application, the ratio of adhesive present to fiber surface available/open area, represented as the adhesive coverage index, is between a lower limit and a higher limit.

According to the invention, the absorbent structure may be mechanically post-treated. For example, the absorbent structure may be introduced into a nip and/or subjected to mechanical post treatments such as, for example, embossing, perforating, brushing, creping, thermo-mechanical bonding (including ultrasonic bonding), aperturing and the like. In an aspect of the present invention, the thin, substantially continuous and uniform layer of adhesive that coats at least a portion of the individual exposed fiber surfaces of the liquid permeable cover has a sufficiently low basis weight to avoid causing sticking, adhesive buildup and relating problems when the post treatment or treatments are carried out.

Desirably, the absorbent structure is mechanically apertured. The mechanical aperturing may be pin-aperturing utilizing a pin or protuberance roll and a female roll.

The liquid permeable nonwoven cover is desirably composed of a matrix of fibrous material. Furthermore, the liquid permeable cover is desirably selected from spunbonded materials, bonded-carded webs and the like.

The at least one layer of at least one liquid management material is desirably selected from bonded-carded webs, air-laid webs, meltblown fiber webs, spunbonded filament webs, hydraulically entangled fiber webs and combinations thereof.

The present invention also encompasses an apertured absorbent structure that includes: (1) a liquid permeable cover made up of a matrix of fibrous materials where the matrix has upper and lower surfaces and a plurality individual exposed fiber surfaces at or adjacent with each surface of the matrix; (2) at least one layer of at least one liquid management material having a upper and lower surface; (3) an adhesive system that joins at least a portion of the lower surface of the liquid permeable cover and the upper surface of the liquid management material; and (4) a series of apertures, defined by the liquid permeable cover and the liquid management material, which extend through the liquid permeable cover and into at least a portion of the liquid management system.

The adhesive system is a thin, substantially continuous coating of adhesive on at least a portion of the plurality of individual exposed fiber surfaces. The overall amount of adhesive is low enough to avoid formation of a hydrophobic barrier layer or liquid impermeable layer between the permeable cover and the liquid management material. The adhesive is also maintains the liquid permeable cover and liquid management material in position to define the individual apertures.

The present invention also encompasses a method of making an absorbent structure that may include the following steps: (1) providing at least a first fibrous material with an upper and lower surface and a second fibrous material with an upper and lower surface; (2) joining the lower surface of the first fibrous material and the upper surface of the second fibrous material with an adhesive to form an absorbent structure; and (3) mechanically post-treating the absorbent structure.

According to the method, the thin, substantially continuous layer of adhesive material is applied at a basis weight sufficiently low to avoid forming a hydrophobic layer between the cover and the liquid management material. This may be accomplished utilizing hot melt adhesives and slot coating techniques. However, aqueous adhesives and label-type adhesives (i.e., adhesives used to attach labels such as, for example, pressure sensitive label adhesives) may be used.

The method of the present invention further contemplates mechanical post-treatment steps such as, for example, embossing, perforating, brushing, creping, thermo-mechanical bonding (including ultrasonic bonding), aperturing and the like. The present invention has been found to work particularly well when practiced in connection with processes to mechanically aperture absorbent structures. More particularly, the present invention has been found to work particularly well when practiced in connection with processes to mechanically aperture absorbent structures having relatively low densities and multi-layer structures in which the layers have different densities and/or different responses to mechanical forces (e.g., different resiliencies, Poisson's ratios or the like).

Another aspect of the present invention relates to the method of maintaining an aperture in a fibrous laminate which may include the steps of: (1) providing at least a first fibrous material with an upper and lower surface and a second fibrous material with an upper and lower surface; (2) joining the lower surface of the first fibrous material and the upper surface of the second fibrous material with an adhesive to form a fibrous laminate; and (3) aperturing the fibrous laminate such that at least one aperture passes through at least one of the fibrous materials and into the other fibrous material such that the adhesive that joins the tow fibrous materials is adapted to maintain the tow fibrous materials in position to define individual apertures.

In an embodiment of the invention, the absorbent structure includes: (1) a liquid permeable cover having a first surface area; (2) at least one layer of at least one liquid management material having a second surface area that is less than the first surface area; and (3) an adhesive system joining the liquid permeable cover and the liquid management material.

### DEFINITIONS

“Disposable” includes being disposed of after use and not intended to be washed and reused.

“Layer” when used in the singular can have the dual meaning of a single element or a plurality of elements.

“Liquid” means a non-particulate substance and/or material that flows and can assume the interior shape of a container into which it is poured or placed.

“Liquid communication” means that liquid is able to travel from one layer to another layer, or one location to another within a layer.

“Longitudinal” means having the longitudinal axis in the plane of the article and is generally parallel to a vertical plane that bisects a standing wearer into left and right body halves when the article is worn. The “transverse” axis lies in the plane of the article generally perpendicular to the longitudinal axis, i.e., so that a vertical plane bisects a standing wearer into front and back body halves when the article is worn.

“Conjugate fibers” refers to fibers that have been formed from at least two polymers extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75125, 50/50, 25/75 or any other desired ratios. The fibers may also have shapes such as those described in U.S. Pat. Nos. 5,277,976 to Hogle et al., and 5,069,970 and 5,057,368 to Largman et al., hereby incorporated by reference in their entirety, which describe fibers with unconventional shapes.

“Biconstituent fibers” refers to fibers that have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein, the term “machine direction” or MD means the length of a fabric in the direction in which it is produced. The term “cross machine direction” or CD means the width of fabric, i.e. a direction generally perpendicular to the MD.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 35 microns. The fibers may also have shapes such as those described in U.S. Pat. Nos. 5,277,976 to Hogle et al., 5,466,410 to Hills and 5,069,970 and 5,057,368 to Largman et al., which describe fibers with unconventional shapes.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers that may be continuous or discontinuous, are generally smaller than 10 microns in average diameter, and are generally tacky when deposited onto a collecting surface.

As used herein, “Airlaying” refers to a well-known process by which a fibrous nonwoven layer can be formed. In the airlaying process, bundles of small fibers having typical lengths ranging from about 3 to about 52 millimeters are separated and entrained in an air supply and then deposited onto a forming screen, usually with the assistance of a vacuum supply. The randomly deposited fibers then are bonded to one another using, for example, hot air or a spray adhesive. Examples of airlaying technology can be found in U.S. Pat. Nos. 4,494,278, 5,527,171, 3,375,448 and 4,640,810.

As used herein, the term “coform” means a process in which at least one meltblown diehead is arranged near a chute through which other materials are added to the web while it is forming. Such other materials may be pulp, superabsorbent or other particles, natural polymers (for example, rayon or cotton fibers) and/or synthetic polymers (for example, polypropylene or polyester) fibers, for example, where the fibers may be of staple length. Coform processes are shown in commonly assigned U.S. Pat. Nos. 4,818,464 to Lau and 4,100,324 to Anderson et al. Webs produced by the coform process are generally referred to as coform materials.

“Bonded carded web” refers to webs that are made from staple fibers that are sent through a combing or carding unit, which opens and aligns the staple fibers in the machine direction to form a generally machine direction-oriented fibrous nonwoven web. The web is bonded by one or more of several known bonding methods.

Bonding of nonwoven webs may be achieved by a number of methods; powder bonding, wherein a powdered adhesive is distributed through the web and then activated, usually by heating the web and adhesive with hot air; pattern bonding, wherein heated calender rolls or ultrasonic bonding equipment are used to bond the fibers together, usually in a localized bond pattern, though the web can be bonded across its entire surface if so desired; through-air bonding, wherein air which is sufficiently hot to soften at least one component of the web is directed through the web; chemical bonding using, for example, latex adhesives that are deposited onto the web by, for example, spraying; and consolidation by mechanical methods such as needling and hydroentanglement.

An intake/distribution layer is a material which can wick menstrual fluid a distance of 1.2 cm to about 15.25 cm (0.5 to 6 inches) in one hour when one end of the material is placed in an infinite reservoir of menstrual simulant.

“Co-aperture” refers to a material which has been apertured, as well as a process of aperturing, wherein two or more materials are apertured together. The apertures extend from top to bottom of the material and are essentially aligned with each other. Co-aperturing can join the materials either temporarily or permanently through entanglement, physical bonding or chemical bonding. It is preferred that co-aperturing be carried out at ambient temperatures, not at elevated temperatures.

“Personal care product” means diapers, training pants, absorbent underpants, adult incontinence products, swim wear, bandages and other wound dressings, and feminine hygiene products.

“Feminine hygiene products” means sanitary napkins and pads.

“Target area” refers to the area or position on a personal care product where an insult is normally delivered by a wearer.

### TEST METHODS

Material Caliper (Thickness):

The caliper of a material is a measure of thickness and is measured at 0.05 psi (3.5 g/cm²) with a Starret-type bulk tester, in units of millimeters.

Density:

The density of the materials is calculated by dividing the weight per unit area of a sample in grams per square meter (gsm) by the material caliper in millimeters (mm) at 0.05 psi (3.5 g/cm²) and multiplying the result by 0.001 to convert the value to grams per cubic centimeter (g/cc). A total of three samples would be evaluated and averaged for the density values.

Triple Intake Test Procedure:

The objective of this test is to determine differences between materials and/or materials, composites or systems of material composites in the rate of intake when 3 fluid insults are applied, with time allowed for fluid to distribute in the material(s) between insults.

Equipment needed:

2 acrylic rate blocks.

P-5000 pipette with RC-5000 tips and foam pipette insert.

Small beaker

Menses simulant (made according to directions below), warmed in bath for 30 minutes or more

Small spatula (stirrer)

Bench liner

2 stopwatches

1-2 timers

Gauze squares for cleaning simulant

Procedure: Lay out sample composites according to materials testing plan.

Components are as follows:

Top: Cover

Middle: Capillarity fabric

Bottom: Retention Layer

Weigh each layer dry, record weight. Put materials back in 3-layer composite.

Weigh a dry blotter, record weight and also mark weight on blotter.

Place acrylic rate block in middle of sample composite.

Calibrate pipette:

Weigh a small empty beaker on the balance.

Set pipette to 2 mis.

Draw simulant into pipette.

Deliver simulant from pipette into beaker.

If balance indicates 2 grams of simulant was delivered, setting is correct.

If more or less than 2 grams was delivered, decrease or increase the setting and repeat adjusting pipette and weighing the amount of stimulant delivered until 2 grams is delivered.

Simulant handling:

Remove simulant from the refrigerator 30 minutes to 1 hour before using and warm in water bath. Before cutting bag nozzle, massage the bag between hands for a few minutes to mix the simulant, which will have separated in the bag. Cut the bag tubing and pour simulant needed into a small beaker. Stir slowly with a small spatula to mix thoroughly. Return bag to the refrigerator if you do not anticipate using all of it. Return bag to water bath if more will be used during the day.

Test:

Step 1: Center acrylic rate block with funnel on sample. Insult sample composite with 2 mis. simulant, using stopwatch to measure the time from the start of the insult until the fluid is absorbed beneath the cover material. Leave rate block in place for 9 minutes, (use timer). For first sample, after 9 minutes remove the rate block and weigh each layer of the sample. Record the weight. (After 3 minutes timing of the first sample, start testing a second sample going through the same steps.)

Step 2: For the first sample, repeat Step 1 a second time.

Step 3: For the first sample, repeat Step 1 a third time.

Analysis: The fluid loading in each component is calculated as weight after insult subtracted from the weight before insult. The insult time is a direct measurement of time for absorption. Smaller values of intake time refer to a more absorbent sample with larger values of intake time refer to a less absorbent sample.

Capacity

Capacity was measured using the dunk and drip capacity test method. Menses simulant was used as the test fluid. The sample size was modified to a 5.7 cm (2.25″) diameter circle. The weight of each sample was recorded. The sample was immersed in a bath of simulant until equilibration, in this case 9 minutes. The sample was removed from the bath and hung vertically at a height of 10.5 cm (12 inches) using a small clip for 10 minutes. The sample was weighed and the weight was recorded. The capacity was determined by subtracting the before weight from the after weight. The capacity in grams/gram was determined by dividing the capacity in grams by the dry weight of the sample.

Horizontal Capillary Wicking Test Procedure:

The objective of this test it to determine the horizontal wicking capability of a material as it pulls fluid from a infinite reservoir.

Equipment needed: Horizontal wicking stand, menses simulant prepared as described below, ruler, timer.

Procedure:

Cut materials to 1″ (2.54 cm) width and desired length.

Fill reservoir in horizontal wicking apparatus with menses simulant.

Place one end of the material in the simulant and lay the rest of the material on the wicking apparatus.

Start the timer.

Measure the distance wicked at a given time, or the time to wick to a given distance.

Flat System Testing Procedure

The purpose of this procedure is to determine the fluid handling characteristics of various absorbent systems through analysis of stain length, saturation capacity, and the fluid loading of the system components. The equipment required includes hourglass-shaped acrylic plates (with a 0.25 inch hole in the center) weighing approximately 330 grams, syringes, one-eighth inch I.D. Tygon tubing, pipette pump, menses simulant, and a laboratory balance (accurate to 0.00 g).

Samples to be tested are cut to a desired shape (currently 1.5 inches by 5.5 inches for fluid intake/distribution layers or capillarity fabrics, 1.75 inches by 5.5 inches for transfer delay layers, and 200 mm long hourglass shape for retention layers). The 5.5 inch layers are marked into 1.1 inch sections and the pad layer is marked into sections corresponding to the marks on the 5.5 inch layers when they are centered on the pad layer. Each component is weighed and the weight recorded. The individual components are assembled into a desired component system maintaining the marked sections aligned and one end is labeled as the top. Syringes are filled with menses simulant and Tygon tubing attached to the syringes. The syringes are placed in a pipette pump which is programmed to deliver a given amount of simulant, currently 30 cc syringes dispensing a specified amount of simulant (usually 10 ml) in one hour. With the open ends of the tubing placed in a beaker, the tubing is primed by running the pump until all air is out of the tubing and simulant is exiting the tubing at the insult end. The component systems to be tested are placed near the pipette pump and a two inch by six inch piece of 25 gsm, 10d BCW is placed on top of the center of the system over which an acrylic plate is placed, also centered on top of the system. The free end of one tubing is inserted into the hole in the acrylic plate and the pipette pump started to begin the insults. At the end of the insult period, the tubing and acrylic plates are removed. The BCW is then carefully removed without moving the underlying layers and discarded. Each layer is then individually weighed and the weight recorded. Then, beginning at the end labeled as the top, each marked section is cut and weighed. The stain length for each layer is measured and recorded and the data entered into a spreadsheet for graphing and analysis. The fluid loading (g/g) is calculated by dividing the amount of fluid absorbed in a material by the dry weight of the material. The fluid saturation is calculated by dividing the fluid loading by the stain length.

Demand Absorbency Wicking Capability:

The objective of this test is to determine the fluid handling characteristics of various absorbent systems through analysis of stain length, saturation capacity, and fluid loadings of the system components.

Equipment needed: Hourglass-shaped acrylic plates (with 0.25″ (6.35 mm) hole in the center) weighing approximately 330 grams; syringes; 1/8 inch (3.175 mm) internal diameter (ID) tubing (e.g. Tygon®); pipette pump; menses simulant prepared as described below; laboratory balance (accurate to 0.00 g).

Procedure:

1. Cut components to desired shape; 1.5 inches (3.8 cm) by 6.0 inches (15.2 cm) for intake/distribution layers, 3.0 inches (7.6 cm) by 6.0 inches for spunbond nonwoven fabric transfer delay and perimeter layers.

2. Mark 6.0 inch layers into 1.2 inch (3 cm) sections. If the perimeter layer is oval, mark into sections corresponding to the marks on the intake/distribution strip when centered on the perimeter layer.

3. Weigh each component and record the weight.

4. Assemble the individual components into the desired absorbent system keeping the marked sections aligned. Label one end as the top.

5. Fill the syringes with menses simulant and attach tubing to syringes.

6. Place the syringes in the syringe pump.

7. Program the size of the syringe into the syringe pump.

8. Program the pump (currently using 30 cc syringes dispensing at a rate of 10 ml. per hour.

9. With the open ends of the tubing placed in a beaker, prime tubing by running pump until all air is out of tubing and simulant is exiting the tubing at the open end.

10. Place the component systems to be tested near the syringe pump, place a 2 inch (5.1 cm) by 6 inch (approximately) piece of 25 gsm, 10 denier bonded carded web material on the top layer of the absorbent system to prevent wicking on the acrylic plate, and place an acrylic plate centered on the top of the system.

11. Insert the open end of one tubing into the hole in the acrylic plate. Repeat for the remaining systems to be tested.

Testing:

1. Start the pipette pump to begin the insult.

2. Ad 3 mis. of menses simulant at a rate of 10 mis per hour.

3. After 3 mis have been insulted into the product, add weights to the acrylic plate to achieve a pressure of 0.08 psi.

4. Continue the insults for another 5 mis, so that a TOTAL of 8 mis is insulted.

5. At the end of the insult period, remove the tubing and acrylic plates. Carefully remove the bonded carded web without moving the underlying layers and discard it.

6. Take photos of the component system and layers and print them.

7. Weigh each layer individually and record the weight.

8. Beginning at the end labeled as the top, cut and weigh the first marked sections and the weight. Repeat for remaining sections and layers.

9. Measure and record the stain length for each layer.

10. Enter the data in a spreadsheet for graphing and analysis.

Preparation of Menses Simulant:

The artificial menses liquid used in the testing was made according to U.S. Pat. No. 5,883,231 from blood and egg white by separating the blood into plasma and red cells and separating the white into thick and thin portions, where “thick” means it has a viscosity after homogenization above about 20 centipoise at 150 sec⁻¹, combining the thick egg white with the plasma and thoroughly mixing, and finally adding the red cells and again thoroughly mixing. A more detailed procedure Follows:

Defibrinated swine blood, is separated by centrifuging at 3000 rpm for 30 minutes, though other methods or speeds and times may be used if effective. The plasma is separated and stored separately, the buffy coat removed and discarded and the packed red blood cells stored separately as well. It should be noted that the blood must be treated in some manner so that it may be processed without coagulating. Various methods are known to those skilled in the art, such as defibrinating the blood to remove the clotting fibrous materials, the addition or anti-coagulant chemicals and others. The blood must be non-coagulating in order to be useful and any method which accomplishes this without damaging the plasma and red cells is acceptable.

Jumbo chicken eggs are separated, the yolk and chalazae discarded and the egg white retained. The egg white is separated into thick and thin portions by straining the white through a 1000 micron nylon mesh for about 3 minutes, and the thinner portion discarded. The thick portion of egg white, which is retained on the mesh, is collected and drawn into a 60 cc (cm³) syringe, which is then placed on a programmable syringe pump and homogenized by expelling and refilling the contents five times. The amount of homogenization is controlled by the syringe pump rate of about 100 mL/min, and the tubing inside diameter of about 0.12 inches. After homogenizing the thick egg white has a viscosity of about 20 centipoise at 150 sec⁻¹ and is then placed in the centrifuge and spun to remove debris and air bubbles at about 3000 rpm for about 10 minutes.

After centrifuging, the thick, homogenized egg white, which contains ovamucin, is added to a 300 cc FENWAL® Transfer pack container using a syringe. Then 60 cc of the swine plasma is added to the FENWAL® Transfer pack container. The FENWAL® Transfer pack container is clamped, all air bubbles removed, and placed in a Stomacher lab blender where it is blended at normal (or medium) speed for about 2 minutes. The FENWAL® transfer pack container is then removed from the blender, 60 cc of swine red blood cells are added, and the contents mixed by hand kneading for about 2 minutes or until the contents appeared homogenous. A hematocrit of the final mixture should show a red blood cell content of about 30 weight percent and generally should be at least within a range of 28-32 weight percent for artificial menses made according to this Example. The amount of egg white is about 40 weight percent.

The ingredients and equipment used in the preparation of artificial menses are readily available. Below is a listing of sources for the items used, though of course other sources may be used providing they are approximately equivalent.

Blood (swine): Cocalico Biologicals, Inc., 449 Stevens Rd., Reamstown, Pa. 17567, (717) 336-1990.

Fenwal® Transfer pack container, 300 mL, with coupler, code 4R2014: Baxter Healthcare Corporation, Fenwal Division, Deerfield, Ill. 60015.

Harvard Apparatus Programmable Syringe Pump model no. 55-4143: Harvard Apparatus, South Natick, Mass. 01760.

Stomacher 400 laboratory blender model no. BA 7021, serial no. 31968: Seward Medical, London, England, UK.

1000 micron mesh, item no. CMN-1000-B: Small Parts, Inc., PO Box 4650, Miami Lakes, Fla. 33014-0650, 1-800-220-4242.

Hemata Stat-II device to measure hemocrits, serial no. 1194Z03127: Separation Technology, Inc., 1096 Rainer Drive, Altamont Springs, Fla. 32714.

Rate Block Intake Test

This test is used to determine the intake time of a known quantity of fluid into a material and/or material system. The test apparatus consists of a rate block 10 as shown in FIG. 1. A 4″×4″ piece of absorbent 14 and cover 13 are die cut. The specific covers are described in the specific examples. The absorbent used for these studies was standard and consisted of 250 g/m² airlaid made of 90% Coosa 0054 and 10% HC T-255 binder. The total density for this system was 0.10 g/cc. The cover 13 was placed over the absorbent 14 and the rate block 10 was placed on top of the two materials. 2 mL of a menses simulant was delivered into the test apparatus funnel 11 and a timer started. The fluid moved from the funnel 11 into a channel 12 where it was delivered to the material or material system. The timer was stopped when all the fluid was absorbed into the material or material system as observed from the chamber in the test apparatus. The intake time for a known quantity of known fluid was recorded for a given material or material system. This value is a measure of a material or material systems absorbency. Typically, five to ten repetitions were performed, and average intake time was determined.

Rewet Test

This test is used to determine the amount of fluid that will come back to the surface when a load is applied. The amount of fluid that comes back through the surface is called the “rewet” value. The more fluid that comes to the surface, the larger the “rewet” value. Lower rewet values are associated with a dryer material and, thus, a dryer product. In considering rewet, three properties are important: (1) intake, if the material/system does not have good intake then fluid can rewet, (2) ability of absorbent to hold fluid (the more the absorbent holds on to the fluid, the less is available for rewet), and (3) flowback, the more the cover prohibits fluid from coming back through the cover, the lower the rewet. In our case, we evaluated cover systems where the absorbent was maintained constant and, thus, we were only concerned with properties (1) and (3), intake and flowback, respectively.

A 4″×4″ piece of absorbent and cover was die cut. The absorbent used for these studies was standard and consisted of a 250 g/m² airlaid made of 90% Coosa 0054 and 10% HC T-255 binder. The total density for this system was 0.10 g/cc. The cover was placed over the absorbent and the rate block was placed on top of the two materials. In this test, 2 mL of menses simulant are insulted into the rate block apparatus and are allowed to absorb into a 4″×4″ sample of the cover material which is placed on top of a 4″×4″ absorbent piece. The fluid is allowed to interact with the system for one minute and the rate block rests on top of the materials. The material system cover and absorbent are placed onto a bag filled with fluid. A piece of blotter paper is weighed and placed on top of the material system. The bag is traversed vertically until it comes into contact with an acrylic plate above it, thus pressing the whole material system against the plate blotter paper side first. The system is pressed against the acrylic plate until a total pressure of 1 psi is applied. The pressure is held fixed for three minutes, after which the pressure is removed and the blotter paper is weighed. The blotter paper retains any fluid that was transferred to it from the cover/absorbent system. The difference in weight between the original blotter and the blotter after the experiment is known as the “rewet” value. Typically, five to ten repetitions of this test were performed, and average rewet was determined.

Intake/Staining Test

An intake/staining test was developed which enables the stain size, intensity, and fluid retention in components to be observed with fluid flow rate and pressure. Menses simulant was used as the test fluid. A 4″×4″ piece of absorbent and cover were die cut. The absorbent used for these tests was standard and consisted of a 250 g/m² airlaid made of 90% of Coosa 0054 and 10% HC T-255 binder. The total density for this system was 0.10 g/cc. A material system, cover and core measuring 4″×4″, was placed underneath an acrylic plate with an 1/8 inch diameter hole bored into the center. A piece of ⅛ inch tubing was connected to the hole with a fitting. Menses simulant was delivered to the sample using a syringe pump at a specified rate and for a specified volume. The pump was programmed to deliver a total volume of 1 mL to the samples, where the samples were under pressures of 0 psi, 0.0078 psi, and 0.078 psi. These pressures were applied using a weight which was placed on top of the acrylic plates and distributed evenly. The flow rate of the pump was programmed to deliver fluid at a rate of 1 mL/sec. The stain size for the cover materials was measured manually, and the amount of fluid in each component of the system was measured by weight before and after absorption of the fluid. The stain intensity was evaluated qualitatively by comparison of samples. Staining information was recorded using a digital camera and could be further analyzed with image analysis.

Permeability

Permeability is obtained from a measurement of the resistance by the material to the flow of liquid. A liquid of known viscosity is forced through the material of a given thickness at a constant flow rate and the resistance to flow, measured as a pressure drop is monitored. Darcy's Law is used to determine permeability as follows:

Permeability=[flow rate×thickness×viscosity/pressure drop] Equation (1) where the units are:

| | |
|---|---|
| permeability: | cm² or darcy 1 darcy = 9.87 × 10⁻⁹ cm² |
| flow rate: | cm/sec |
| viscosity: | pascal-sec |
| pressure drop: | pascals |

The apparatus consists of an arrangement wherein a piston within a cylinder pushes liquid through the sample to be measured. The sample is clamped between two aluminum cylinders with the cylinders oriented vertically. Both cylinders have an outside diameter of 3.5″, an inside diameter of 2.5″ and a length of about 6″. The 3″ diameter web sample is held in place by its outer edges and hence is completely contained within the apparatus. The bottom cylinder has a piston that is capable of moving vertically within the cylinder at a constant velocity and is connected to a pressure transducer that capable of monitoring the pressure of encountered by a column of liquid supported by the piston. The transducer is positioned to travel with the piston such that there is no additional pressure measured until the liquid column contacts the sample and is pushed through it. At this point, the additional pressure measured is due to the resistance of the material to liquid flow through it.

The piston is moved by a slide assembly that is driven by a stepper motor. The test starts by moving the piston at a constant velocity until the liquid is pushed through the sample. The piston is then halted and the baseline pressure is noted. This corrects for sample buoyancy effects. The movement is then resumed for a time adequate to measure the new pressure. The difference between the two pressures is the pressure due to the resistance of the material to liquid flow and is the pressure drop used in Equation (1). The velocity of the piston is the flow rate. Any liquid whose viscosity is known can be used, although a liquid that wets the material is preferred since this ensures that saturated flow is achieved. The measurements disclosed herein were carried out using a piston velocity of 20 cm/min, mineral oil (Peneteck Technical Mineral Oil manufactured by Penreco of Los Angeles, California) of a viscosity of 6 centipoise.

Alternatively, permeability can be calculated from the following equation:

Permeability=0.051 *R*(1 -Porosity)*(Porosity/(1 -Porosity))^{2 75} Equation (2)

where R=fiber radius and

Porosity=1-(web density/fiber density) Equation (3)

Reference for Equation (2) can be found in the article “Quantification of Unidirectional Fiber Bed Permeability” by J. Westhuizen and J. P. Du Plessis in the Journal of Composite Materials, 28(7), 1994. Note that the equations show that permeability can be determined if fiber radius, web density and fiber density are known.

Conductance is calculated as permeability per unit thickness and gives measure of the openness of a particular structure and, thus, an indication of the relative ease at which a material will pass liquid. The units are darcies/mil.

Adhesive Coverage Index

The adhesive coverage index relates the measures of adhesive present in a sampled area of material to the amount of fibrous surface area (amount of open area) in the same sampled area of material.

The amount of adhesive present in a sampled area is measured with image analysis techniques. The sample is prepared for imaging using a 72% sulfuric acid treatment.

Data is acquired using a Quantimet 600 IA System and the QUIPS routine ‘PERCOV2.’ The optical configuration includes a SONY 3CCD video camera, a 40 mm-EI Nikkor lens with a 20-mm extension tube, four flood lamps, and a white background. Data is accumulated from a minimum of four fields-of-view.

### DETAILED DESCRIPTION

Generally speaking, the invention relates to an absorbent structure that includes: (1) a liquid permeable cover made up of a matrix of fibrous materials where the matrix has upper and lower surfaces and a plurality individual exposed fiber surfaces at or adjacent with each surface of the matrix; (2) at least one layer of at least one liquid management material having a upper and lower surface; and (3) an adhesive system that joins at least a portion of the lower surface of the liquid permeable cover and the upper surface of the liquid management material.

According to the invention, the adhesive system is a thin, substantially continuous coating of adhesive on at least a portion of the plurality of individual exposed fiber surfaces. The overall amount of adhesive is low enough to avoid interfering with liquid transport through the liquid permeable cover and into the liquid management material. In other words, the amount of adhesive is low enough to essentially prevent formation of a hydrophobic barrier layer or liquid impermeable layer between the permeable cover and the liquid management material.

While the adhesive system is desirably a hot-melt adhesive, other adhesives such as, for example, aqueous adhesives and label-type adhesives may be used. Generally speaking, the adhesive should avoid form globules or globs on the surface of the permeable cover and should instead form a thin, generally continuous coating or film. The adhesive is desirably applied in such a generally thin, uniform film by utilizing slot coat techniques. Other application techniques that provide a thin, uniform continuous coating on the permeable cover material surface may be used.

For example, conventional adhesives and adhesive coating or application techniques may be adapted to the practice of the present absorbent pad. Exemplary adhesives include, but are not limited to, hot-melt garment construction adhesives used in the manufacture of personal care products (e.g., diapers, incontinence products, feminine care products) such as those available under the designations: DF-561 0; 34-5610; 434-5563; 34-5606; 134-5551; 34-5582; IL-88; 34-5561; 1716; and 518-3312 from National Starch, Bridgewater, N.J. Other suitable hot-melt construction adhesives are available under the designation D-9105; D-3950; D-8370; JM-1004-A; and D-9105-ZP from HB Fuller, St. Paul, Minn. Even more suitable hot-melt construction adhesives are available under the designation L-8507; L-8007; H-2457; H-1 091 from Ato Findley Inc., of Wauwatosa, Wis.

The continuous layer of adhesive overlaying the permeable cover material desirably has a basis weight of 7.5 gsm or less. For example, layer of adhesive may have a basis weight of 4 gsm or less. As another example, the layer of adhesive may have a basis weight of 2.5 gsm or less. As yet another example, the layer of adhesive may have a basis weight of 1.5 gsm or less. As a further example, the layer of adhesive may have a basis weight of 1.0 gsm or less.

In one embodiment of the present invention, the continuous layer of adhesive effectively coats the plurality of individual exposed fiber surfaces such that in the area of adhesive application, the ratio of adhesive present to fiber surface available/open area, represented as the adhesive coverage index, is between about 5 and to about 25.

According to the invention, the absorbent structure may be mechanically post-treated. For example, the absorbent structure may be introduced into a nip and/or subjected to mechanical post treatments such as, for example, embossing, perforating, brushing, creping, thermo-mechanical bonding (including ultrasonic bonding), aperturing and the like. In an aspect of the present invention, the thin, substantially continuous and uniform layer of adhesive that coats at least a portion of the individual exposed fiber surfaces of the liquid permeable cover has a sufficiently low basis weight to avoid causing sticking, adhesive buildup and relating problems when the post treatment or treatments are carried out.

Desirably, the absorbent structure is mechanically apertured. The mechanical aperturing may be pin-aperturing utilizing a pin or protuberance roll and a female roll. Such coaperturing can be accomplished by a number of processes including a matched roll pin aperturing process or a pattern/anvil roll pin aperturing process.

The matched roll pin aperturing process is widely used to aperture single layer materials. This process may be used to aperture a multilayer structure where the apertures extend through all of the layers of the multilayer cover system. In this process, a low permeability material is unwound on top of a high permeability material and the two materials are then passed over a bowed bar to an aperturing unit and through a nip.

The nip consists of a pair of two matched rolls, one male and one female. The male roll is characterized by a series of pins arranged in a specific pattern extending from a roll. The female roll is characterized by a series of recesses into which the pins of the male should fit such that the two rolls are mateable. The two rolls are driven with matched gearing to insure registration. The two rolls may be heated with electrical heaters. When the materials pass through the nip, the apertures are created through temperature and pressure. Through the cooperative rotation of the male and female rolls, the pins of the male roll extend into the materials and displace the materials to the extent of the pin diameters/perimeter. The displaced material is then compressed and densified between the surface of the pins of the male roll and the surface of the recesses of the female roll. As the materials continue passing through the cooperatively rotating male and female rolls, the pins are removed from the material and the resultant aperture is left in the materials. The adhesive that is used to laminate the materials helps maintain the aperture by providing additional bonding in the densified material found in the perimeter of the aperture. After aperturing, the materials are wound onto a roll.

Such an aperturing process is distinct from a punching process where material is removed from the material web rather than the displacement and densification of material found with aperturing.

Equipment used for pin aperturing of the material may have two rolls positioned one on top of the other. In one case, the top roll (male roll) may have plates in which pins having a diameter of 0.081 inches and providing a defined pattern can be fixed. Other patterns can be used consisting of pins of different size and shape. The bottom roll (female roll) has recesses in its structure in which the pins can fit. The separation on the two rolls can be varied depending upon the material being processed. Heat may be applied to both rolls in order to aid the process. The temperature of the top roll may be in the range of 100° F. (23.5° C.) to 500° F. (118° C.). The temperature of the bottom roll may also in the range of 100° F. to 500° F. if heat is applied to the bottom roll. Alternatively and/or additionally, heat or other energy may be applied directly to the assembly to be apertured. Material is processed at the rate of about 10 to about 2000 feet per minute. Tension may placed onto the material using a driven unwind. If tension is placed on the material, the material may relax after aperturing and generate puckers.

Description of an exemplary system for aperturing and/or co-aperturing 20 webs and web assemblies is described in U.S. patent application Ser. No. 60/336,097, entitled “System For Aperturing And Co-Aperturing Webs And Web Assemblies” by James M. Keane et al., assigned to the assignee of the present application and filed on even date herewith. The contents of that application are incorporated herein by reference in their entirety.

In an exemplary process, a continuous strip of liquid management material (e.g., air laid liquid distribution material) is fed into a rotary die module where the absorbent material is radial end cut forming an oval shaped component. A discrete piece that may be, for example, 190 mm long, is placed at product pitch on a continuous liquid permeable cover material (e.g., a spunbond web) that is traveling at machine velocity. For example, with a final pad length of 238 mm and a 190 mm×37 mm discrete piece of liquid management material centered on the pad, a gap of 48 mm exists between successive liquid management material components. A thin film of adhesive is slot coated onto the spunbond layer (less than 7.5 gsm) with dimensions slightly smaller than that of the discrete piece.

The assembly composed of the spunbond layer, discrete air laid liquid management material, and adhesive then passes through a rotary aperture module (aperturing system with a pin roll and female roll) where an aperture is formed (starting from the cover material side) through both the cover material and the liquid management material.

On the liquid management material side of the aperture, the liquid management material is elongated in the Z direction. The displaced liquid management material is compacted by the pin and female recess geometry forming a dense structure around the aperture hole. An important feature for maintaining the aperture quality throughout the product life cycle is the densification of the liquid management material around the perimeter of the aperture. In those situations where the liquid management material contains a large portion of cellulose fibers, it is generally desirable for the cellulose fibers to become densified. The thin, continuous coating of adhesive that laminates the liquid permeable cover to the liquid management material further aids in maintaining aperture quality by providing additional bonding in the densified area of material found at the perimeter of the aperture.

The liquid permeable nonwoven cover is desirably composed of a matrix of fibrous material. Furthermore, the liquid permeable cover is desirably selected from spunbonded materials, bonded-carded webs and the like. An exemplary spunbonded material is a 20 gram per square meter (basis weight) polypropylene nonwoven cover material available from Kimberly-Clark Corporation. Other cover materials that are considered include woven and nonwoven materials and laminates including, but not limited to, meltblown fiber webs, fabric-like films, nonwoven-film laminates and the like.

The at least one layer of at least one liquid management material is desirably selected from bonded-carded webs, air-laid webs, meltblown fiber webs, spunbonded filament webs, hydraulically entangled fiber webs and combinations thereof. The liquid management material may be homogeneous or may have strata composed of air-laid staple length fibers, air-laid fluff cellulose fibers, air-laid chemically modified cellulose fibers, hydrogel fibers and combinations thereof. In an aspect of the invention, the generally stratified layers of the first liquid absorbent component comprises at least two layers of a fibrous nonwoven web. Desirably, at least one of the layers of a fibrous nonwoven web is selected from bonded-carded webs, air-laid webs, meltblown fiber webs, spunbonded filament webs, hydraulically entangled fiber webs and combinations thereof.

In an aspect of the invention, the liquid management material may further include the first liquid absorbent component may have strata composed of air-laid staple length fibers, air-laid fluff cellulose fibers, air-laid chemically modified cellulose fibers, hydrogel fibers and combinations thereof. In an aspect of the invention the generally stratified layers of the first liquid absorbent component comprises at least two layers of a fibrous nonwoven web. Desirably, at least one of the layers of a fibrous nonwoven web is selected from bonded-carded webs, air-laid webs, meltblown fiber webs, spunbonded filament webs, hydraulically entangled fiber webs and combinations thereof.

Examples of suitable materials that are useful in the present invention include several multifunctional air laid materials available from Buckeye Technologies, Inc. (Memphis, Tenn.). One such material comprises an air-formed multi-strata web comprised of polyester (PET) fibers, fluff cellulose fibers and chemically modified cellulose fibers that are formed onto a carrier tissue sheet, which is then bonded with a combination of a PET/polyethylene bi-component binder fiber and an ethylvinyl alcohol-based latex emulsion.

Binders can be used to help provide mechanical integrity and stabilization. Binders include fiber, liquid or other binder means which may thermally activated. Desirable binder fibers include those having a relative melting point such as polyolefin fibers. Fibers having a lower melting polymer, like conjugate and biconstituent fibers are desirable. Fibers having a lower melting polymer are generally referred to as “fusible fibers”. By “lower melting polymers” what is meant are those having a glass transition temperature less than about 175 C. It should be noted that the texture of the absorbent web could be modified from soft to stiff through selection of the glass transition temperature of the polymer. Exemplary binder fibers include conjugate fibers of polyolefins, polyamides and polyesters. Exemplary binder fibers include sheath core conjugate fibers available from KoSa Inc. (Charlotte, N.C.) under the designation T-255 (Merge 34821 A) and T-256 or Copolyester designation, though many suitable binder fibers are known to those skilled in the art, and are available by many manufacturers such as Chisso and Fibervisions LLC of Wilmington, Del. KoSa has developed a suitable co-polyester binder fiber as a sheath core application and is known by designation T254 (low melt COPET). A suitable liquid binder is KYMENE® 557LX available from Hercules Co. of Wilmington, Del. Other suitable liquid binders include ethylene vinyl acetate emulsion polymers sold by National Starch and Chemical Company (Bridgewater, N.J.) under the tradename DUR-O-SET® ELITE® series (including ELITE® 33 and ELITE® 22). Air Products Polymers and Chemicals sells other suitable binder fibers under the name AIRFLEX®.

Synthetic fibers include those made from polyamides, polyesters, rayon, acrylics, superabsorbents, TENCEL® regenerated cellulose and any other suitable synthetic fibers known to those skilled in the art. Synthetic fibers may also include kosmotropes for product degradation.

Many polyolefins are available for fiber production, for example polyethylenes such as Dow Chemical's ASPUN® 6811A liner low density polyethylene, 2553 LLDPE and 25355 and 12350 high density polyethylene are such suitable polymers. The polyethylenes have melt flow rates, respectively, of about 26, 40, 25 and 12. Fiber forming polypropylenes include Exxon Chemical Company's ESCORENE® PD 3445 polypropylene and Montell Chemical Co.'s PF304. Many other polyolefins are also available.

Particularly preferred materials for this application include polyesters, which may range in size or denier from 3 to 25 denier, and having various cross-sections including round, pentalobal, helical crimped, etc. Such fibers have been developed by KoSa, Inc. with a durably wettable finish and are known by designation of fiber denier followed by polymer type and cross section. Examples would include 8 dpf, T-224 (High Void); 8 dpf, T-224 (trilobal); 15 dpf T-224 (round); 10 dpf T-224 (round); 6 dpf T-224 (round) and 3 dpf T-224 (round).

Natural fibers include wool, cotton, flax, hemp and wood pulp. Wood pulps include standard softwood fluffing grade such as CR-1654 (U.S. Alliance Pulp Mills, Coosa, Ala.). Pulp may be modified in order to enhance the inherent characteristics of the fibers and their processability. Curl may be imparted to the fibers by methods including chemical treatment or mechanical twisting. Curl is typically imparted before crosslinking or stiffening. Pulps may be stiffened by the use of crosslinking agents such as formaldehyde or its derivatives, glutaraldehyde, epichlorohydrin, methylolated compounds such as urea or urea derivatives, dialdehydes, maleic anhydride, non-methylolated urea derivatives, citric acid or other polycarboxylic acids. Some of these agents are less preferable than others due to environmental and health concerns. Pulp may also be stiffened use of heat or caustic treatments such as mercerization. Examples of these types of fibers include NHB416 which is a chemically crosslinked southern softwood pulp fibers which enhances wet modulus, available from the Weyerhaeuser Corporation of Tacoma, Wash. Other useful pulps are debonded pulp (NF405) and non-debonded pulp (NB416) also from Weyerhaeuser. HPZ3 from Buckeye Technologies, Inc of Memphis, Tenn., has a chemical treatment that sets in a curl and twist, in addition to imparting added dry and wet stiffness and resilience to the fiber. Another suitable pulp is Buckeye HPF2 pulp and still another is IP SUPERSOFT® from International Paper Corporation. Suitable rayon fibers are 1.5 denier Merge 18453 fibers from Tencel Incorporated of Axis, Ala.

As a more detailed example, an exemplary material suitable for the first liquid absorbent component is available from Buckeye Technologies, Inc. under the trade designation Buckeye Unicore 8001. This air-formed or air-laid multi-strata material may have a total basis weight in the range from about 120 to about 300 (desirably between about 210 and 240) grams per square meter (gsm) and an overall density ranging from about 0.06 to about 0.10 grams per cubic centimeter (g/cm3). Exemplary multi-strata materials may have a top or uppermost layer of about 25 to about 45 gsm that contains latex bonded polyester fibers ranging from about 6 to about 15 denier per fiber (dpf) and which desirably make up about 20% of the total basis weight of the strata in the first liquid absorbent component. An immediately adjacent layer may be about 35 to about 70 gsm and may contain cellulose fiber joined with a binder fiber. The cellulose may be mercerized cellulose that is thermally bonded utilizing a bicomponent polyester/polyethylene binder fiber. This layer may desirably make up about 30% of the total basis weight of the strata. The multi-strata material may further include a layer that is about 35 to about 100 gsm and may include a compressible cellulose and binder fiber of the same or similar type as in the adjacent layer. This other layer may make up about 40% of the total basis weight of the strata. These layers may be formed on or supported by a carrier tissue that may range from about 10 to about 20 gsm and may desirably make up about 5 percent of the total basis weight of the strata.

Other examples of suitable coherent, flexible matrices including stratified layers of fibrous material that may be used as the first liquid absorbent component can be found in International Publication Number WO 00/74620. According to that publication, the terms “strata” and “stratum” refer to the layered regions which make up a unitary structure. The strata of the unitary structure is not an assembly or laminate of preformed layers forming a multilayered structure. Instead, the unitary structure is constructed by assembling the strata in a continuous, manner. Airlaid technology is described as the method for assembling the strata of the unitary structure.

Another example of a suitable coherent, flexible matrix including stratified layers of fibrous material that may be used as the first liquid absorbent component has two strata or layers. The first layer is an air-laid structure having a basis weight of about 50 gsm and containing 85%, by weight, polyester fibers—denier per filament that is bonded together with about 15%, by weight, of a conventional latex binder suitable for personal care products. The second layer is an air-laid structure having a basis weight of about 150 gsm and containing about 90%, by weight, cellulose fluff and about 10%, by weight, bi-component binder fibers composed of a polyester core and a polyethylene or polyethylene-like sheath that softens or melts when heat is applied to dry the latex binder and thermally activate the binder fibers. This specific combination layers had an overall thickness of about 1.6 millimeters.

Yet another example of a suitable material is an air-laid web composed of about an air-laid structure having a basis weight of about 150 gsm and containing about 90%, by weight, cellulose fluff and about 10%, by weight, bi-component binder fibers composed of a polyester core and a polyethylene or polyethylene-like sheath that softens or melts when heat. The material may have a basis weight ranging from about 175 gsm to about 250 gsm.

In an aspect of the invention, one or more of these layers may further include particulate materials. These particulate materials may be superabsorbent materials (also referred to as such as hydrogel materials).

The present invention also encompasses an apertured absorbent structure that includes: (1) a liquid permeable cover made up of a matrix of fibrous materials where the matrix has upper and lower surfaces and a plurality individual exposed fiber surfaces at or adjacent with each surface of the matrix; (2) at least one layer of at least one liquid management material having a upper and lower surface; (3) an adhesive system that joins at least a portion of the lower surface of the liquid permeable cover and the upper surface of the liquid management material; and (4) a series of apertures, defined by the liquid permeable cover and the liquid management material, which extend through the liquid permeable cover and into at least a portion of the liquid management system.

The adhesive system is a thin, substantially continuous coating of adhesive on at least a portion of the plurality of individual exposed fiber surfaces. The overall amount of adhesive is low enough to avoid formation of a hydrophobic barrier layer or liquid impermeable layer between the permeable cover and the liquid management material. The adhesive is also maintains the liquid permeable cover and liquid management material in position to define the individual apertures.

The present invention also encompasses a method of making an absorbent structure that may include the following steps: (1) providing at least a first fibrous material with an upper and lower surface and a second fibrous material with an upper and lower surface; (2) joining the lower surface of the first fibrous material and the upper surface of the second fibrous material with an adhesive to form an absorbent structure; and (3) mechanically post-treating the absorbent structure.

According to the method, the thin, substantially continuous layer of adhesive material is applied at a basis weight sufficiently low to avoid forming a hydrophobic layer between the cover and the liquid management material. This may be accomplished utilizing hot melt adhesives and slot coating techniques. However, aqueous adhesives and label-type adhesives (i.e., adhesives used to attach labels such as, for example, pressure sensitive label adhesives) may be used.

The method of the present invention further contemplates mechanical post-treatment steps such as, for example, embossing, perforating, brushing, creping, thermo-mechanical bonding including ultrasonic bonding, aperturing and the like. The present invention has been found to work particularly well when practiced in connection with processes to mechanically aperture absorbent structures. More particularly, the present invention has been found to work particularly well when practiced in connection with processes to mechanically aperture absorbent structures having relatively low densities and multi-layer structures in which the layers have different densities and/or different responses to mechanical forces (e.g., different resiliencies, Poisson's ratios or the like).

In an embodiment of the invention, a continuous strip of liquid permeable cover material (e.g., a continuous spunbond cover material) is traveling at constant machine velocity, V1. The rotary die cutting module, rotating at a velocity, V2 (slower than machine velocity), radially end cuts a liquid management material (e.g., an airlaid distribution layer) producing discrete components which are then transferred to the continuous spunbond cover material at product pitch. The velocity difference between the cutter and the web causes a high degree of machine direction (MD) placement variability of the component.

As a process aid, hot melt adhesive is placed on the continuous web prior to transferring the component from the knife roll of the rotary cutter to the continuous web. The adhesive, preferably pulsed so that the adhesive and discrete component are timed together helps reduce MD placement variability of the component.

According to the present invention, a substantially continuous thin film adhesive layer is deposited between the liquid management material and cover material. The approximate dimensions of the adhesive range from 125 to 250 mm in length (dependent on the liquid management material length) and approximately 37 mm wide. These dimensions are less than the dimensions of the liquid management material (e.g., liquid distribution layer).

For nonwoven materials, the level of adhesive should remain below 7.5 gsm to prevent the adhesive from bleeding through the cover material. Desirably the adhesive is in the form of a slot coat of adhesive just short of the full liquid management material length. Again, the dimensions of the adhesive should be smaller than that of the distribution layer and the add-on should be less than 7.5 gsm.

By keeping the adhesive between the cover and distribution layer, the adhesive that was previously exposed to the nip situation is eliminated and the aperture hole quality is improved. It is necessary to maintain the low add-on of adhesive to prevent problems with the product functionality. At this low level of adhesive, no problems were found with product performance.

Products with coaperturing and a pin stripe adhesive pattern had intake rates measured as described above of approximately 29 seconds with 6 mL of Z-Date. The products tested with the thin film of adhesive also had intake rates of approximately 28 seconds. However, there is a high potential for problems to occur at higher adhesive levels, due to a formation of a hydrophobic film layer between the cover and distribution layer of an absorbent product and the bleed-through mentioned earlier.

While slot coating adhesive applications are thought to work best, it is contemplated that other adhesive application/patterns can be used including, for example, swirl, meltblown, bead systems and the like. Alternatively and/or additionally, aqueous adhesive systems, pressure sensitive adhesive systems may also be used.

It is believed that applying a substantially continuous layer of adhesive between the liquid permeable cover and a liquid management material would typically be thought to substantially impair fluid intake between the liquid permeable cover and the absorbent material. However, it has been found that adhesive add-on at the prescribed levels applied in substantially solid or continuous form directly between the liquid management material and the liquid permeable cover does not negatively influence liquid intake when the two components are subsequently co-apertured.

More particularly, it has been found that the prescribed adhesive add-on levels does not negatively impact the intake of complex liquids such as menses.

In an aspect of the present invention, the substantially continuous or solid layer of adhesive between the cover and the liquid management material enhances the aesthetics of the aperture by having a higher percentage of the liquid management material bonded to the cover material. For example, during aperturing of an airlaid pulp joined to a spunbond web, the relatively resilient spunbond web remains joined to the air-laid web in those locations where the combination is apertured.

The substantially continuous application of adhesive is thought to help prevent the nonwoven material from recovering from the in-plane dislocation that occurs from aperturing and or embossing. Other adhesive application methods listed above can be used to attach cover and absorbent layers for maintaining machine direction placement. However, random or open patterns such as those described in U.S. Pat. Nos. 4,069,822; 4,147,580 and 4,573,986 will leave non-bonded areas between the absorbent and nonwoven allowing the nonwoven to recover from the dislocation causing the materials to separate after the aperturing process.

Another aspect of the present invention relates to the method of maintaining an aperture in a fibrous laminate which may include the steps of: (1) providing at least a first fibrous material with an upper and lower surface and a second fibrous material with an upper and lower surface; (2) joining the lower surface of the first fibrous material and the upper surface of the second fibrous material with an adhesive to form a fibrous laminate; and (3) aperturing the fibrous laminate such that at least one aperture passes through at least one of the fibrous materials and into the other fibrous material such that the adhesive that joins the tow fibrous materials is adapted to maintain the tow fibrous materials in position to define individual apertures.

In an embodiment of the invention, the absorbent structure includes: (1) a liquid permeable cover having a first surface area; (2) at least one layer of at least one liquid management material having a second surface area that is less than the first surface area; and (3) an adhesive system joining the liquid permeable cover and the liquid management material.

While this invention has been described as having a preferred embodiment, it will be understood that it is capable of further modifications. It is therefore intended to cover any variations, equivalents, uses, or adaptations of the invention following the general principles thereof, and including such departures from the present disclosure as come or may come within known or customary practice in the art to which this invention pertains and falls within the limits of the appended claims.

We claim:

1. An absorbent structure comprising:
a liquid permeable cover comprising a matrix of fibrous material, the matrix having upper and lower surfaces and a plurality of individual exposed fiber surfaces at or adjacent with each surface of the matrix;
at least one layer of a liquid management material having an upper and lower surface; and
an adhesive system joining at least a portion of the lower surface of the liquid permeable cover and the upper surface of the liquid management material, the adhesive system comprising
a substantially continuous coating of adhesive on at least a portion of the plurality of individual exposed fiber surfaces such that the overall amount of adhesive is low enough to avoid interfering with liquid transport from the cover material to the liquid management material.

2. The absorbent structure of claim 1, wherein the substantially continuous coating of adhesive on individual exposed fiber surfaces is present such that in the area of adhesive application the adhesive coverage index is between about 5 and about 25.

3. The absorbent structure of claim 1, wherein the absorbent structure is mechanically post-treated.

4. The absorbent structure of claim 3, wherein the absorbent structure is mechanically apertured.

5. The absorbent structure of claim 1, wherein the substantially continuous coating of adhesive on individual exposed fiber surfaces is applied with slot coat techniques.

6. The absorbent structure of claim 1, wherein the liquid permeable cover is selected from spunbonded materials, bonded-carded webs and the like.

7. The absorbent structure of claim 1, wherein at least one layer of the liquid management material is selected from bonded-carded webs, air-laid webs, meltblown fiber webs, spunbonded filament webs, hydraulically entangled fiber webs and combinations thereof.

8. A personal care product incorporating the absorbent structure of claim 1.

9. An apertured absorbent structure comprising:
a liquid permeable cover comprising a matrix of fibrous material, the matrix having upper and lower surfaces and a plurality of individual exposed fiber surfaces at or adjacent with each surface of the matrix;
at least one layer of a liquid management material having an upper and lower surface;
an adhesive system joining at least a portion of the lower surface of the liquid permeable cover and the upper surface of the liquid management material, the adhesive system comprising
a substantially continuous coating of adhesive on at least a portion of the plurality of individual exposed fiber surfaces such that the overall amount of adhesive is low enough to avoid interfering with liquid transport from the cover material to the liquid management material; and
where the liquid permeable cover and liquid management material define a series of apertures which extend through the liquid permeable cover and into at least a portion of the liquid management material
where the adhesive system is adapted to maintain the liquid permeable cover and liquid management material in position to define individual apertures.

10. The absorbent structure of claim 9, wherein the substantially continuous coating of adhesive on individual exposed fiber surfaces is present such that in the area of adhesive application the adhesive coverage index is between about 5 and about 25.

11. The absorbent structure of claim 9, wherein the substantially continuous coating of adhesive on individual exposed fiber surfaces is applied with slot coat techniques.

12. The absorbent structure of claim 9, wherein the liquid permeable cover is selected from spunbonded materials, bonded-carded webs and the like.

13. The absorbent structure of claim 9, wherein at least one layer of the liquid management material is selected from bonded-carded webs, air-laid webs, meltblown fiber webs, spunbonded filament webs, hydraulically entangled fiber webs and combinations thereof.

14. A personal care product incorporating the absorbent structure of claim 9.

15. A method for production of an improved absorbent structure comprising the steps:
providing at least a first fibrous material with an upper and lower surface and a second fibrous material with an upper and lower surface;
joining the lower surface of the first fibrous material and the upper surface of the second fibrous material with an adhesive to form an absorbent structure;
mechanically post treating the absorbent structure.

16. The method of claim 15 where the step of mechanically post treating the absorbent structure further consists of aperturing the absorbent structure such that at least one aperture passes through at least one of the fibrous materials into the other fibrous material and the adhesive that joins the two fibrous materials is adapted to maintain the two fibrous materials in position to define individual apertures.

17. The method of claim 15, wherein the first fibrous material is a liquid permeable cover and the second fibrous material is a liquid management material.

18. The method of claim 15, wherein the fibrous materials are joined by applying an adhesive in a substantially continuous thin layer on at least one of either the lower surface of the first fibrous layer or the upper surface of the second fibrous material and contacting the lower surface of the first fibrous layer and upper surface of the second fibrous layer.

19. The method of claim 18, wherein the substantially continuous thin layer of adhesive is applied utilizing a slot coat technique.

20. The method of claim 18, where the substantially continuous coating of adhesive on individual exposed fiber surfaces is present such that in the area of adhesive application the adhesive coverage index is between about 5 and about 25.

21. The method of claim 15, wherein the overall amount of adhesive applied is low enough to avoid interfering with liquid transport from the cover material to the liquid management material.

22. An absorbent structure produced by the method of claim 15.

23. A personal care product incorporating an absorbent structure produced by the method of claim 15.

24. A method of maintaining an aperture in a fibrous laminate consisting of the steps:
providing at least a first fibrous material with an upper and lower surface and a second fibrous material with an upper and lower surface;
joining the lower surface of the first fibrous material and the upper surface of the second fibrous material with an adhesive to form a fibrous laminate;
aperturing the fibrous laminate such that at least one aperture passes through at least one of the fibrous materials into the other fibrous material and the adhesive that joins the two fibrous materials is adapted to maintain the two fibrous materials in position to define individual apertures
